(19) United States
(12) Patent Application Publication
(10) Pub. No.: US 2021/0045943 A1
(43) Pub. Date: Feb. 18, 2021

(54) Adjustable Absorbent Article

(71) Applicant: Better Made Hemp, LLC, Northville, MI (US)

(72) Inventor: Alexandra Blasius, Northville, MI (US)

(21) Appl. No.: 16/994,081

(22) Filed: Aug. 14, 2020

Related U.S. Application Data
(60) Provisional application No. 62/887,082, filed on Aug. 15, 2019.

(51) Int. Cl. A61F 13/80; A61F 13/56; A61F 13/514; B32B 33/00

(52) U.S. Cl. CPC A61F 13/80; A61F 13/5611; A61F 2013/51443; B32B 33/00; A61F 13/51401

(57) ABSTRACT

An adjustable absorbent article includes a center portion including a plurality of layers for absorption of fluid. The adjustable absorbent article also includes a side portion delineated from the center portion by at least one perforation line and positioned adjacent the center portion, the at least one perforation line extends through the adjustable absorbent article allowing the absorbent article to be adjustable by a wearer. The side portion is removable from the center portion by tearing along the at least one perforation line for allowing a wearer to adjust the absorbent article. The adjustable absorbent article further includes an adhesive portion disposed on the center portion for adhering to an undergarment of the wearer. The center portion, side portion, adhesive portion or any combinations thereof comprises hemp.

### CROSS-REFERENCE TO RELATED APPLICATIONS

This application claims priority to and all the advantages of U.S. Provisional Application 62/887,082 filed on Aug. 15, 2019, the entire content of which is incorporated herein by reference in its entirety.

### BACKGROUND

Conventional absorbent articles are used to absorb and hold bodily exudates (e.g. urine, menses). There are many different types of absorbent articles, which may include diapers, pants, sanitary napkins, and tampons to prevent leakage of bodily exudates. There have been efforts to prevent any leakage of menstrual blood to a wearer's undergarments due to misalignment caused by poor adhesion, improper layering of material, and/or non-durable material. It is often desirable to prevent any leakage to a wearer's undergarment or unwanted areas of the wearer's body and, at the same time, provide an optimal fit for the wearer.

In addition to the above requirements, absorbent articles also need to meet many absorbent capacity requirements. During the menstrual cycle, a substantial amount of bodily fluids exit the body and the absorbent article the wearer uses needs to ensure the undergarments are not stained and, worse, leak beyond the undergarments. Some absorbent articles include multiple layers of material that is capable of quickly absorbing fluids and subsequently passing the fluids to the next layer to retain the fluids, whereas others concentrate on the type of material of the absorbent article to provide enough absorbency.

A wearer may purchase different types of absorbent articles to fit different types of undergarments and clothing including swimsuits. Undergarments come in different shapes, lengths, and sizes. For example, the wearer might buy a thinner, slimmer shaped absorbent article to fit slimmer undergarments (e.g. thongs) and a thicker, wider shaped absorbent article to fit more full-sized undergarments. Buying different types of absorbent articles to fit different undergarments can become expensive. Further, it may be an inconvenience and a waste if the wearer decides to change undergarments and is required to throw away unused absorbent articles in order to fit another undergarment. Another disadvantage is that the absorbent article may shift out of place due to the twisting, distorting, and stretching of the absorbent article due to the movements of the wearer. Lastly, conventional absorbent articles are single use products and thrown away with one use, which can harm the environment.

To this end, there remains a need for an improved absorbent article.

### SUMMARY OF THE DISCLOSURE AND ADVANTAGES

An adjustable absorbent article includes a center portion including a plurality of layers for absorption of fluid. The adjustable absorbent article also includes a side portion delineated from the center portion by at least one perforation line and positioned adjacent the center portion, the at least one perforation line extends through the adjustable absorbent article allowing the absorbent article to be adjustable by a wearer. The side portion is removable from the center portion by tearing along the at least one perforation line for allowing a wearer to adjust the absorbent article. The adjustable absorbent article further includes an adhesive portion disposed on the center portion for adhering to an undergarment of the wearer. The center portion, side portion, adhesive portion or any combinations thereof includes hemp.

Accordingly, the adjustable absorbent article may be used with different types of undergarments, remains secured to a wearer's undergarment, and is biodegradable and environmentally friendly.

### BRIEF DESCRIPTION OF THE DRAWINGS

Referring now to the drawings, exemplary illustrations are shown in detail. Although the drawings represent schematic configurations, the drawings are not necessarily to scale and certain features may be exaggerated to better illustrate and explain an innovative aspect of an illustrative configuration. Further, the exemplary illustrations described herein are not intended to be exhaustive or otherwise limiting or restricting to the precise form and configuration shown in the drawings and disclosed in the following detailed description.

Advantages of the present disclosure will be readily appreciated, as the same becomes better understood by reference to the following detailed description when considered in connection with the accompanying drawings.

FIG. 1A is a top perspective view of an exemplary full-size configuration of the adjustable absorbent article, according to one configuration.

FIG. 1B is a bottom perspective view of the adjustable absorbent article of FIG. 1A.

FIG. 2 is an exploded top view of the adjustable absorbent article of FIG. 1A including a plurality of layers.

FIG. 3 is a top perspective view illustrating removal of a side portion of the absorbent article via tearing of the side portion along a perforated line.

FIG. 4 is a top perspective view illustrating removal of another side portion of the absorbent article via tearing of the side portion along a perforated line.

FIG. 5A is top perspective view of an exemplary full-size configuration of the adjustable absorbent article, according to another configuration.

FIG. 5B is a bottom perspective view of the adjustable absorbent article of FIG. 5A.

FIG. 6 is a top perspective view of a first profile illustrating removal of a side portion of the absorbent article via tearing of the side portion along a perforated line.

FIG. 7 is a top perspective view illustrating removal of another side portion, converting from the first profile to a second profile.

FIG. 8 is a top perspective view illustrating removal of another side portion, converting from the second profile to a third profile.

FIG. 9 is a top perspective view illustrating removal of another side portion, converting from the second profile to a fourth profile.

FIG. 10 is a top perspective view of an exemplary thong configuration of the adjustable absorbent article being attached to a wearer's undergarment.

### DETAILED DESCRIPTION

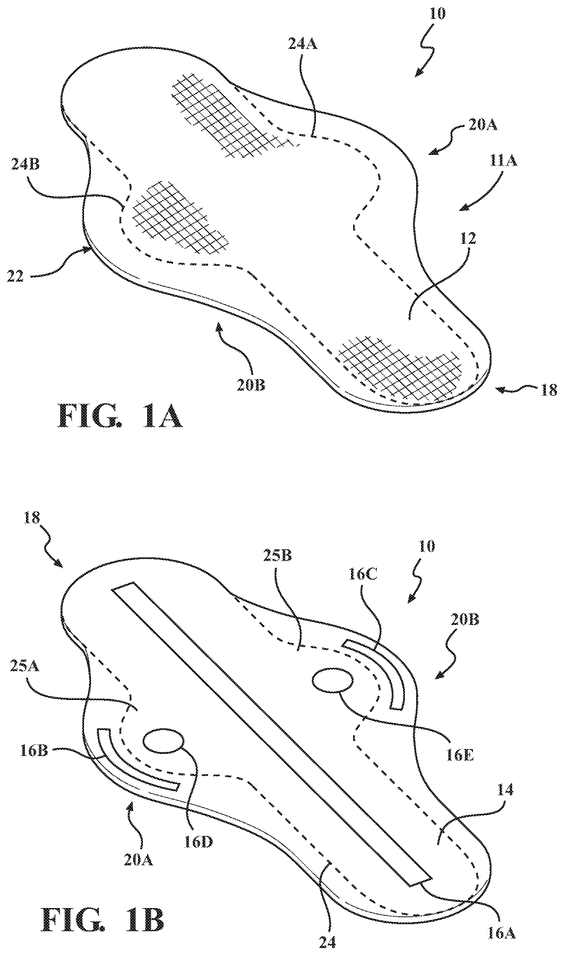

An adjustable absorbent article 10 is generally shown in FIGS. 1A and 1B. A wearer may adhere the adjustable absorbent article 10 to an undergarment or any clothing, including swimsuits, for absorption and collection of bodily exudates. In the configurations shown throughout, the adjustable absorbent article 10 is shown as an absorbent pad. Although not shown, it will be appreciated that the adjustable absorbent article 10 may be a diaper, pants, sanitary napkin, or party-liner.

With reference to FIG. 1A, the absorbent article 10 includes a center portion 18 including a plurality of layers 22 for absorption of fluid. The adjustable absorbent article 10 also includes a side portion 18 delineated from the center portion 18 by at least one perforation line 24 and positioned adjacent the center portion 18. It is to be appreciated that the adjustable absorbent article 10 may have one side portion 20 or may have more than one side portion 20. The at least one perforation line 24 extends through the adjustable absorbent article 10 allowing the absorbent article 10 to be adjustable by a wearer. The side portion 18 is removable from the center portion 18 by tearing along the at least one perforation line 24 for allowing a wearer to adjust the absorbent article 10. The adjustable absorbent article 10 further includes an adhesive portion 16 disposed on the center portion 18 for adhering to an undergarment of the wearer.

Typically, the adjustable absorbent article 10 includes a top surface 12, a bottom surface 14 opposite the top surface 12 and an adjustable profile 11. It is to be appreciated that the center portion 18 may include the top surface 12 and the bottom surface 14. The adhesive portion 16 may be provided and disposed on the bottom surface 14 for adhering the absorbent article 10 to an undergarment of a wearer. Of course, the adhesive portion 16 can adhere the absorbent article 10 to various other types of clothing, including swimsuits and one-pieces. To use the adjustable absorbent article 10, the wearer may simply remove a protective strip (also referred to as a release liner) from the adhesive portion 16 located on the bottom surface 14 of the adjustable absorbent article 10. Once removed, the wearer may secure the adjustable absorbent article 10 to an undergarment. It is contemplated that there may be a protective strip provided for every adhesive portion 16. The top surface 12 is exposed to the wearer's vaginal/crotch area for collecting any bodily exudates and the bottom surface 14 is adhered to the undergarment for stability during the wearer's movements.

With reference to the one or more side portions 20, the one or more side portions 20 may be detachable or tearable from the center portion 18. In particular, the at least one perforation line 24 may include a plurality of perforations extending through the absorbent article 10, which allows a wearer to tear the one or more side portions 20 from the center portion 18. The perforation line 24 may extend completely or at least partially through the absorbent article 10. As described above, the center portion 18 includes one or more layers for absorption of bodily exudates and may at least partially define the top and bottom surfaces 12, 14 of the absorbent article 10.

At least one of the center portion 18, the side portion 20, and the adhesive portion 16 comprises hemp. Hemp includes, but is not limited to, hemp fiber, hemp oil, hemp paste, hemp extract, hemp probiotic, hemp remedy, hemp essence, and combinations thereof. Hemp will be discussed in further detail below. The center portion 18, the detachable side portion 20, and the adhesive portion 16 are discussed in further detail below. Having at least one of the center portion 18, the side portion 20, and the adhesive portion 16 comprising hemp offers several advantages, including providing a biodegradable and environmentally friendly absorbent article. Additional advantages are described in further detail below.

As mentioned above, the absorbent article 10 has an adjustable profile 11. The absorbent article 10 may be adjusted from the initial profile 11A to a second profile 11B by tearing the one or more detachable side portions 20 along the at least one perforation line 24, which removes the one or more detachable side portions 20 from the absorbent article 10, thereby allowing a user or a wearer to change the profile 11 of the absorbent article 10 as desired. The one or more side portions 20 at least partially defines an initial profile 11A of the adjustable absorbent article 10 and is removably attached to the adjustable absorbent article 10. It is contemplated that the absorbent article 10 may include any number of side portions 20. In one configuration, the one or more detachable side portions 20 are shown as side portions 20A and 20B.

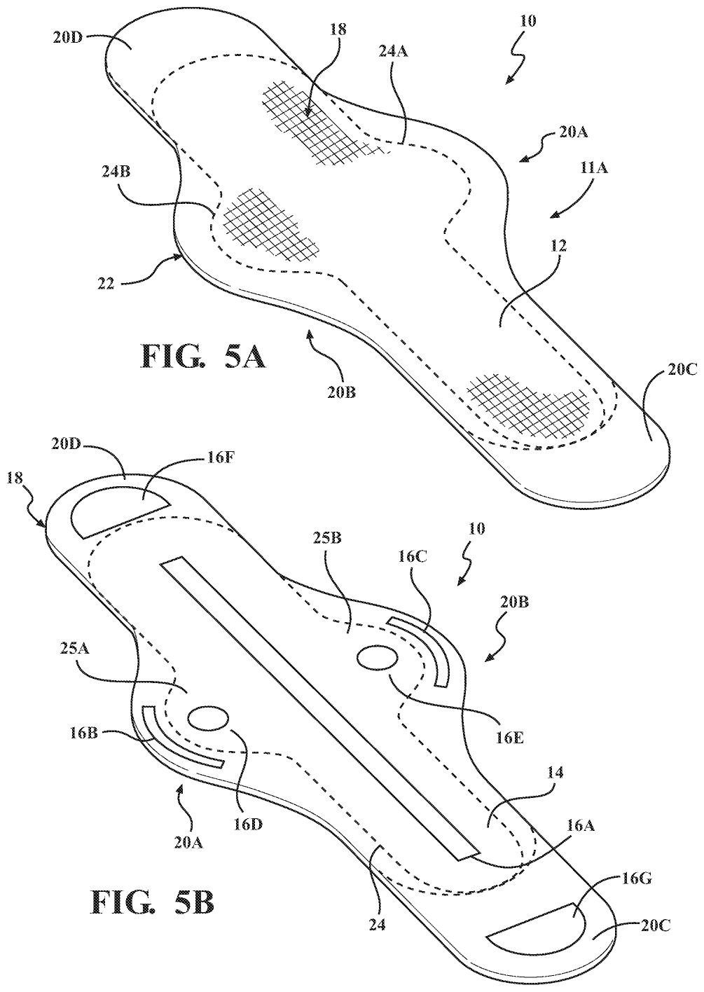

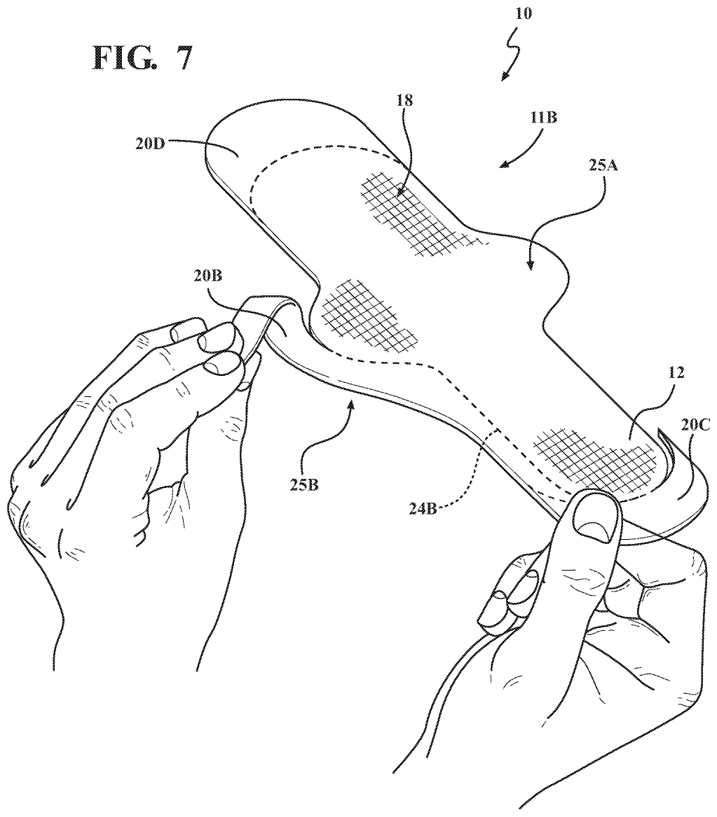

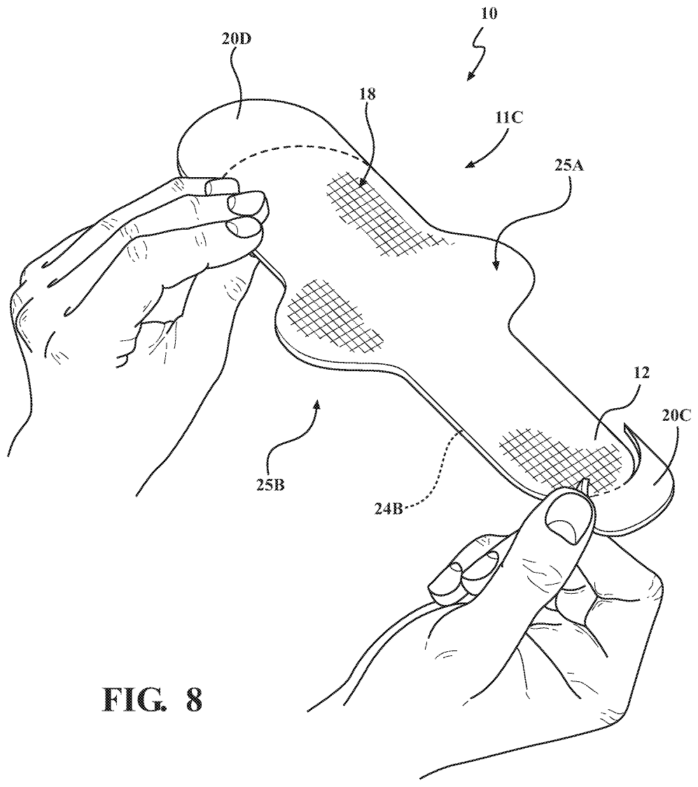

The absorbent article 10 may include two or more side portions 20. The two or more side portions 20 includes a plurality of layers, including at least one impervious layer. With reference to FIGS. 5A and 5B, the side portions are shown as 20A, 20B, 20C, and 20D. In such embodiments, the side portions 20A, 20B, 20C and 20D may be detachable from the center portion 18. The absorbent article 10 is adjusted from an initial profile 11A, as shown in FIG. 5A, to a second profile 11B, as shown in FIG. 7, by tearing the two or more detachable side portions 20 along the at least one perforation line 24, removing the two or more detachable side portions 20 from the absorbent article 10, and thereby allowing a user or a wearer to change the profile 11 of the absorbent article 10 as desired. Further, in these configurations, the absorbent article 10 may be adjusted from the second profile 11B to a third profile 11C, as shown in FIG. 8, by tearing the two or more detachable side portions 20 along the at least one perforation line 24.

Referring again to FIGS. 1A-1B, the absorbent article 10 includes a first side portion 20A and a second side portion 20B. The first and second side portions 20A, 20B are detachable. As shown in FIG. 1B, the adhesive portion 16 is disposed on the bottom surface 14 for adhering the adjustable absorbent article 10 to the undergarment of the wearer. For example, the adhesive portion 16 may be disposed on the entire bottom surface 14 of the adjustable absorbent article 10. Alternatively, the adjustable absorbent article 10 may include a plurality of adhesive portions 16, as shown in FIGS. 1B and 5B. For example, there may be a first adhesive portion 16A disposed on the bottom surface 14 of a center portion 18 and a second adhesive portion 16B disposed on the bottom surface 14 of a side portion 20. In another example, shown in FIG. 1B, a first adhesive portion 16A may be disposed on the bottom surface 14 of the center portion 18, a second and third adhesive portion 16B, 16C disposed on the bottom surface 14 of a winged portion 25B of a first and second side portion 20A, 20B, respectively, and a fourth and fifth adhesive portion 16D, 16E disposed on the bottom surface 14 of a winged portion 25A of the center portion 18, respectively. The adhesive portion 16 may be applied in any suitable manner or pattern including, but not limited to, slots, spirals, spray, coating, and splatters. Further, adhesives may be applied in any suitable amount.

It will be appreciated that the adjustable absorbent article 10 may include any number of portions (adhesive, center, side, wing), shown as A, B, C, and so on. For example, as shown in FIGS. 1A and 1B, the adjustable absorbent article 10 includes the center portion 18, the first side portion 20A, the second side portion 20B, a first perforation line 24A, and a second perforation line 24B. In another example, FIGS. 5A and 5B illustrate the adjustable absorbent article 10 including the center portion 18, the first side portion 20A, the second side portion 20B, the third side portion 20C, and the fourth side portion 20D. Each side portion 20A, 20B, 20C, 20D may be delineated from the center portion 18 by a perforation line 24. The center portion 18 and the side portion(s) 20 are configured to aid in the absorption and collection of bodily fluids, stability of the adjustable absorbent article 10 or any combination thereof.

The center portion 18 and the side portion(s) 20 may each include a winged portion 25. For example, as shown throughout the FIGS., the center portion 18 includes a first winged portion 25A and the first and second side portions 20A, 20B include a second winged portion 25B. In this configuration, the first and second side portions 20A, 20B are positioned along a perimeter of the center portion 18. The first and second side portions 20A, 20B include the second winged portion 25B configured to be shaped around the first winged portion 25A. It will be appreciated that the center portion 18 and the side portion(s) 20 may include any number of winged portions. For example, the first side portion 20A may include a first winged portion and the second side portion 20B may include a second winged portion, separate from the first winged portion of the first side portion 20A.

In configurations wherein the adjustable absorbent article 10 is in the full-size configuration, the wearer may wrap the winged portion 25B under the undergarment to keep the adjustable absorbent article 10 in place and provide extra stability. It will be appreciated that the full-size configuration is typically the initial profile 11A.

In configurations wherein the adjustable absorbent article 10 is in a thong configuration, the wearer may wrap the winged portion 25A of the center portion 18 under the undergarment to keep the adjustable absorbent article 10 in place and provide extra stability. It will be appreciated that the thong configuration is the second profile 11B. The thong configuration is adjusted from the full-size configuration by detaching the side portions 20A, 20B thereby adjusting the absorbent article 10 from the initial profile 11A to the second profile 11B. The adjustability of the absorbent article 10 will be discussed in greater detail below.

The winged portion 25 may be integrated with the center and/or side portions 18, 20 such that the center and/or side portion 18, 20 and the respective winged portions 25A, 25B are a single component. Alternatively, the winged portion 25 may be detachable from the center and/or side portions 18, 20 depending on the wearer's discretion. For example, to provide a more comfortable or optimal fit, the wearer may detach (via tearing, pulling, or the like) a winged portion 25 along a perforation line 24 delineating a side portion 20 and a center portion 18.

As shown throughout the FIGS., the side portion 20 is positioned adjacent the center portion 18. In one configuration, as shown in FIGS. 1A-4, the adjustable absorbent article 10 may include a first side portion 20A and a second side portion 20B. In this configuration, the first and second side portions 20A, 20B are positioned on the left and right side of the center portion 18. In another configuration, as shown in FIGS. 5A-9, the adjustable absorbent article 10 may include a first side portion 20A, a second side portion 20B, a third side portion 20C, and a fourth side portion 20D. In this configuration, the first and second side portions 20A, 20B are positioned on the left and right side of the center portion 18 and the third and fourth side portions 20C, 20D are positioned at an end of the center portion 18 and another end, opposite the first end, of the center portion 18. In another configuration, the adjustable absorbent article 10 may include one side portion 20 connected around the perimeter of the center portion 18. In yet another configuration, the adjustable absorbent article 10 may include a first, second, and third side portion 20 positioned around the perimeter of the center portion 18 to allow for two or more different widths and/or lengths. It will be appreciated that the side portion 20 may be positioned adjacent the center portion 18 in any location along the perimeter of the center portion 18. It will further be appreciated that the side portion 20 may be any length along the perimeter of the center portion 18.

The side portion(s) 20 may include any various sizes and dimensions to be positioned adjacent the center portion 18. For example, the first side portion 20A and the second side portion 20B may include a length of greater than half the length of the center portion 18 and a width of about an inch from the center portion 18. In another example, the side portion 20 may be formed around the center portion 18 and shaped to correspond to the shape of the center portion 18. In this example, the side portion 20 is one piece and is configured to be shaped around the perimeter of the center portion 18. Other sizes and dimensions of the side portion 20 are contemplated.

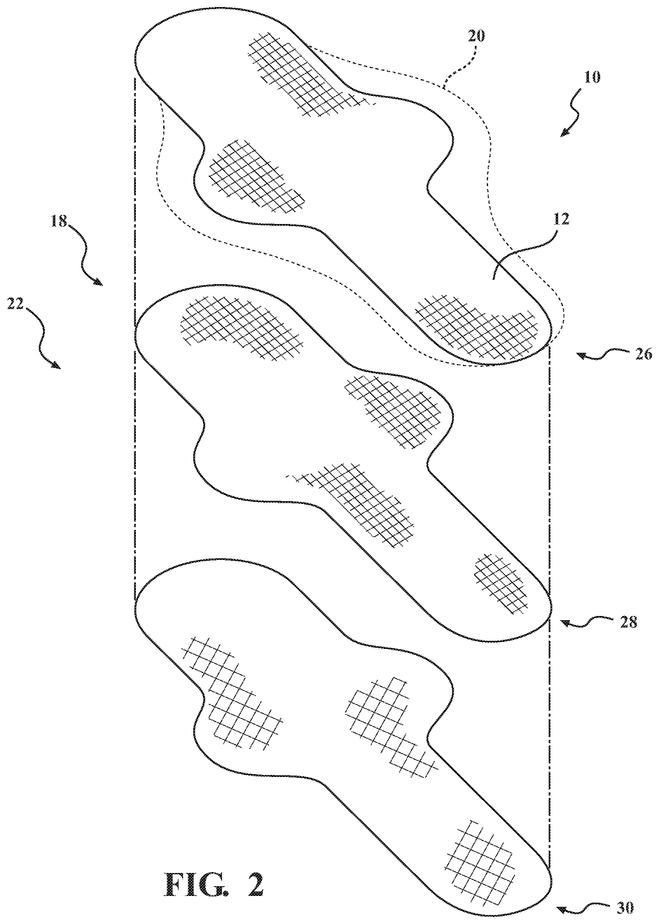

Referring to FIG. 2, the center portion 18 includes the plurality of layers 22 to collect and absorb any bodily exudates, especially menstrual fluids. The top, core, and/or bottom layers 26, 28, 30 may be provided to collect, absorb, retain, and/or stop the bodily exudates. For example, the center portion 18 may include a top layer 26 to collect fluid, a core layer 28 to absorb the fluids, and a fluid impervious bottom layer 30. There can be one or more of the core layer 28. In some examples, one or more of the layers (e.g. the bottom layer 30) is impervious. In such an example, the bottom layer 30 typically does not allow fluid to pass through. In some examples, the impervious layer comprises a biobased polymer. In some examples, the impervious layer comprises a water-dispersible and/or a biodegradable polymer. The bottom layer 30 may include a top surface and a bottom surface opposite the top surface. The adhesive portion is disposed on the bottom surface such that the adhesive portion is configured to face towards the undergarment. Since the bottom layer 30 is the last layer before the undergarment, it is important that the bottom layer 30 does not allow any fluid to pass through, ensuring no leakage to the undergarment or any unwanted area of the wearer.

Each of the plurality of layers may have any number of groups of the combinations of one or more layers of absorbent material and one or more layers of semipermeable material. For example, the core layer 28 may have one or more layers of absorbent material and one or more layers of semipermeable material, followed by a second group of one or more layers of absorbent material and one or more layers of semipermeable material.

It is contemplated that the plurality of layers 22 may be laid loosely, adhered or otherwise removably secured, and/or fixedly attached or secured to the preceding one of the plurality of layers 22, the top layer 26, the bottom layer 30, the core layer 28, and/or any combinations thereof.

In configurations wherein the core layer 28 includes multiple layers, it may be desirable for at least one layer to be continuous to form a web-like structure. For example, to form the web-like structure, several individual fibers or threads may be intertwined or interlaid. It will be appreciated that the web-like structure may be formed by various methods and techniques.

The plurality of layers may include different lengths, widths, and/or thicknesses. For example, the core layer 28 may be thicker than the top layer 26. As mentioned above, the core layer 28 may include one or more layers of material. In another example, one of the multiple layers may extend to the side portion 20. In other words, the side portion 20 may include one or more layers of material. In this configuration, the side portion 20 may aid in the absorption and collection of bodily exudates by comprising absorbent material, similar to the core layer 28. It will be appreciated that the side portion 20 may include any number of layers in a similar manner as the center portion 18. It should also be appreciated that the side portion 20 may include a different number of layers than the center portion 18, e.g. the center portion 18 could include three layers and the side portion could include two layers.

The absorbent article 10 may include any type of natural material, including natural fibers. Use of natural material is advantageous as natural materials are a sustainable resource, as they are renewable, biodegradable, and carbon neutral. Natural material may be used without depleting or damaging the environment. In some configurations, the absorbent article 10 and a packaging assembly may consist of, consist essentially of, or comprise biodegradable material. The absorbent article 10 and/or the packaging assembly may be biodegradable, compostable, or flushable. Prior to use of the absorbent article 10, the wearer may unwrap or unpackage the absorbent article from the packaging assembly and throw away the packaging assembly. Preferred packaging assemblies are made from biodegradable materials which create minimal or no environmental issues. The packaging assembly may include any number of moisture-proof, child-proof, and/or tamper-proof wrappers and/or containers for storage of the absorbent article 10 prior to use. A container may include any number of individually wrapped, via wrappers, absorbent articles 10. The packaging assembly prevents moisture from contacting the absorbent article 10, prevents children from easily accessing the absorbent article 10, and therefore aids in the assurance of shelf-stability and provides an acceptable product prior to actual use. It is appreciated that the absorbent article 10 described herein may be used with the packaging assembly.

The packaging assembly may comprise a biopolymer, a water-dispersible/water-soluble polymer, and/or a biodegradable polymer. The packaging assembly may be made from a combination of thermoplastic materials, especially a blend of water-dispersible polymers and biodegradable polymers to result in disintegrable applicators. In one example, the packaging assembly may comprise a biodegradable polymer. In other examples, the packaging assembly may consist essentially of, or consist of, a biodegradable polymer. The term “consist essentially of,” when used to describe the proportion of a material, suitably means includes at least 90% by weight of the given material, more suitably at least 95% by weight of the given material, and most suitably at least 99% by weight of the given material. It will be appreciated that the absorbent article 10 and/or the packaging assembly may include any amount of biodegradable polymer and/or water-dispersible polymers.

In some configurations, for packaging purposes, a release liner or protective strip can be disposed on the adhesive portion(s) 16 to keep the absorbent article 10 from adhering to packaging surfaces, a user's hands, etc. prior to the use of the absorbent article. In some examples the release liner comprises a biopolymer. The release liner can comprise a water-dispersible and/or a biodegradable polymer. In one example, the release liner comprises a biopolymer, a water-dispersible, and/or a biodegradable polymer with a release coating (e.g. a silicon coating) that allows for easy removal from the adhesive portion(s) 16.

Further, the packaging assembly may include biomaterials made from renewable raw materials, i.e. biopolymers. The packaging assembly may include polymers which are formed from plant-based polymers or polymers formed from renewable resources, e.g. polymers that are not formed with petroleum based feedstocks. The packaging assembly of the absorbent article 10 may include up to 100% by weight of a polymer formed from renewable resources based on a total weight of the packaging assembly. Examples of renewable resources are plants including, but not limited to, sugar cane, beets, corn, potatoes, citrus fruits, woody plants, and cellulosic waste. Using the packaging assembly comprising a renewable resource reduces environmental impact of the absorbent article 10. For example, the packaging assembly may include sugar cane. Sugar cane takes up carbon dioxide during growth and results in a decrease of environmental carbon dioxide versus using polyethylene derived from petroleum-based resources. In another example, the packaging assembly may include hemp. Hemp is biodegradable and takes less time to decompose as compared to fossil-fuel-based material.

It is contemplated that the absorbent article 10 and/or the packaging assembly may be constructed in any blend, composite, shape, or configuration using water-dispersible and/or biodegradable polymers, and any other desired or optional ingredient to provide benefits to the absorbent article 10 or to the materials used in making the absorbent article 10. Such benefits include, but are not limited to, stability, flexibility, resiliency, toughness, strength, workability, and odor control. The optional ingredient may include, but is not limited to, plasticizing agents, antioxidants, slip agents, flow promoters, processing aids, pigments or colorants, mold release agents, coating agents, gelling agents, antistatic agents, dispersing agents, lubricants, surfactants, odor masking agents, opacifying agents such as aluminum oxide, dyes, viscosity modifiers, waxes, elastomers, and mixtures thereof.

It will further be appreciated that the term “biodegradable” as used herein refers to materials that when disposed of after use will physically and/or biologically decompose via known degradation procedures including, but not limited to, aerobic, anaerobic, and microbial digestion processes. The biodegradable materials described herein include those degradable water-insoluble materials that will also physically and/or biologically decompose after disposal.

For example, any component, part, and/or feature may include greater than 30, 40, 50, 60, 70, 80, 90, or 95, % by weight of a water-dispersible and/or biodegradable polymer based on the total weight of the component. The amount of biodegradable polymer may vary outside of the ranges described above. In additional non-limited embodiments, all values and ranges of values within are hereby expressly contemplated.

Any component, part, and/or feature of the absorbent article 10 may include biomaterials comprising or formed with renewable raw materials. The absorbent article 10 may include polymers which are formed from plant-based polymers or polymers formed from renewable resources, e.g. polymers that are not formed with petroleum based feedstocks, which are referred to herein as biopolymers. The absorbent article 10 may include up to 100% by weight of various combinations of biomaterials which are formed with renewable resources based on a total weight of the absorbent article 10. Examples of renewable resources are plants including, but not limited to, sugar cane, beets, corn, potatoes, citrus fruits, woody plants, and cellulosic waste. Using a renewable resource alternative reduces environmental impact.

For example, any component, part, and/or feature may include greater than 30, 40, 50, 60, 70, 80, 90, or 95, % by weight of a biopolymer based on the total weight of the component. The amount of biopolymer may vary outside of the ranges described above. In additional non-limited embodiments, all values and ranges of values within are hereby expressly contemplated. It is contemplated that biomaterials may include, but are not limited to, bio-polymers including hemp, sugar cane, microorganisms, cellulose, starch, and any other polymeric material including polylactic acid, polyhydroxyalkanoate, polybutylene succinate, polyhydroxybutyrate, and polybutylene terephthalate.

A suitable natural material or biomaterial is hemp-based. Hemp is derived from the Cannabis sativa plant species. Cannabis sativa plant is a multi-purpose plant. For example, the plant that may be domesticated for fiber in the stem, processed for oil from the seeds, and grown for other purposes including, but not limited to, medicinal and therapeutic purposes. Specifically, hemp can be grown as a fiber and/or seed. Hemp stalk and seed(s) are harvested to produce products including, but not limited to, textiles, paper, building materials, foods, personal hygiene products and seed cakes. Hemp-based material or biomaterial includes, but is not limited to, hemp fiber, hemp oil, hemp paste, hemp extract, hemp probiotic, hemp remedy, hemp essence and combinations thereof.

In various configurations, the absorbent article 10 may include hemp-based biomaterial in an amount of up to 10, 20, 30, 40, 50, 60, 70, 80, 90, or 100, % by weight based on the total weight of the absorbent article 10. Alternatively, the hemp-based biomaterial can be present in an amount of 1 to 20, 21 to 40, 41 to 60, 61 to 80, 81 to 99, % by weight based on a total weight of the absorbent article 10. The amount of hemp-based biomaterial may vary outside of the ranges described above. In additional non-limited embodiments, all values and ranges of values within are hereby expressly contemplated.

It will be appreciated that, the absorbent article 10 may include one or more hemp-based materials selected from a group consisting of fiber, oil, paste, extract, probiotic, remedy, essence and a combination thereof. It will further be appreciated that the hemp-based material may be a strain selected from the group consisting of a Cannabis indica strain, a Cannabis sativa, a hybrid strain, a high-CBD strain, and a high-THC strain.

The Cannabis sativa plant may produce hemp fibers, hemp oil, and other plant materials. Hemp fibers may be derived from the stalk and hemp oil may be produced from the seed. It will be appreciated that hemp fiber, hemp oil, and any other hemp material may be processed, produced, or the like in any suitable method. It will be understood that other plant material derived from the Cannabis sativa plant are contemplated.

The layers and portions of the absorbent article 10 may include hemp fiber. In some examples, the plurality of layers 22 of the center portion 18, including the bottom layer 30, and the side portion(s) 20, may comprise, consist essentially of, or consist of, a plant-based or natural fiber. In such examples, the plant-based or natural fiber can be selected from hemp, cotton, flax, bamboo, and combinations thereof. Of course, the layers and portions of the adjustable absorbent article 10 can include other types of natural fiber. In some configurations, the absorbent article 10 may include greater than 50, 60, 70, 80, 90, or 95, % by weight plant-based or natural fiber based on a total weight of the absorbent article 10. In other configurations, the absorbent article 10 may include greater than 50, 60, 70, 80, 90, or 95, % by weight hemp fiber based on a total weight of the absorbent article 10. The amount of plant-based or natural fiber (e.g. hemp fiber) may vary outside of the ranges described above. In additional non-limited embodiments, all values and ranges of values within are hereby expressly contemplated.

In some configurations, the plurality of layers 22 of the center portion 18, including the bottom layer 30, and the side portion(s) 20, may consist of, consist essentially of, comprise hemp fiber or hemp-based polymer. Due to the porous nature of hemp fiber, hemp fiber has excellent absorbent properties. The porous nature of hemp fiber allows the absorbent article 10 to readily absorb fluid and to be quick drying.

The hemp fiber and/or thread may be knitted, twisted, weaved. The hemp fiber and/or thread may be bonded in a pre-determined pattern or method to adjacent fibers of the plurality of layers 22 wherein absorption of liquid or fluid by the plurality of layers 22 of the center portion 18 and/or the side portion 20 aids in prevention of fluid leakage.

In some configurations, the top layer 26, the core layer 28, and/or the bottom layer 30 may include greater than 10, 20, 30, 40, 50, 60, 70, 80, 90, or 90, % by weight hemp fiber, based on the total weight of the particular layer. In other configurations, the center portion 18 and/or the side portion 20 may include greater than 10, 20, 30, 40, 50, 60, 70, 80, 90, or 90, % by weight hemp fiber based on the total weight of the center portion 18 and/or the side portion 20. The amount of hemp fiber may vary outside of the ranges described above. In additional non-limited embodiments, all values and ranges of values within are hereby expressly contemplated.

In some configurations, the adhesive portion 16 may comprise biodegradable material. In other configurations, the adhesive portion 16 may consist essentially of or consist of biodegradable material. In one particular example the adhesive portion comprises a hemp-based adhesive. Further, the adhesive portion 16 may include hemp oil, hemp paste, hemp essence, and/or combinations thereof.

In some configurations, hemp oil, hemp paste, hemp essence, and/or combinations thereof may be applied to the top layer 26 of the absorbent article 10 in any suitable manner or pattern including, but not limited to, slots, spirals, spray, coating, and splatters. Moreover, the hemp oil, hemp paste, hemp essence, and/or combinations thereof may be applied in any suitable amount.

As mentioned above, the Cannabis sativa plant may produce hemp oil for technical hemp products. More specifically, the seed(s) may be crushed and processed to produce hemp oil. It will be appreciated that other types of technical hemp products including, but not limited to, oil pants, printing inks, solvents, lubricants, coatings, or any combinations thereof may be applied to the absorbent article 10.

From an environmental perspective, coating systems including excessive amounts of volatile fluids may be harmful to the environment. Additionally, coating systems may be formed from non-renewable resources. The absorbent article 10 may include a coating that is eco-friendly and/or formed from renewable resources. A suitable coating may be hemp-based. Including a hemp-based coating allows the absorbent article 10 to potentially be entirely biodegradable. In preferred configurations, the absorbent article 10 may include a hemp-based coating. In various configurations, the top surface 12 and/or the bottom surface 14 of the adjustable absorbent article 10 may include a coating produced from the plant Cannabis sativa. The coating may be applied to the absorbent article 10 in any manner. Further, the coating may be applied to enhance the aesthetic appeal by accentuating the top and/or bottom surface 12, 14. Other applications of the hemp-based coating are contemplated.

Alternatively, or additionally, the coating may be applied for therapeutic applications. For example, the coating on the top surface 12 may help remedy menstrual cramping as the top surface 12 is exposed to the wearer's body and/or may be in contact with the wearer's skin. The coating may be applied to the top and/or bottom surface 14 using any suitable method. Other components or features of the adjustable absorbent article 10 including, but limited to, the protective strip and a wrapper may also be made from the herbaceous plant of the species Cannabis sativa or the like. For example, the protective strip may consist of, consist essentially of, or comprise bioplastic material, including hemp.

From a therapeutic perspective, natural oils may be extracted from a plant and applied to absorbent articles to alleviate or relieve pains, aches, cramps related to menstruation. Natural oil is an oil obtained from a renewable source. Some natural oils include, but are not limited to, canola oil, castor oil, coconut oil, cottonseed oil, olive oil, palm oil, peanut oil, rapeseed oil, soybean oil, hemp oil, and combinations thereof.

One particularly suitable natural oil is hemp oil, which is derived from the plant Cannabis sativa. In a preferred configuration, the adjustable absorbent article 10 may be infused with oil produced or extracted from the plant Cannabis sativa. For example, the plurality of layers 22 of the center portion 18 and/or the side portions 20 may be infused with hemp oil for therapeutic remedies including, but not limited to, healing, moisturizing, or relieving the wearer of any menstrual pain or cramping. Further, the hemp oil may be infused to result in less odor. Other applications of the hemp oil are contemplated. In another example, hemp oil may be processed to produce a paste for adhering to the bottom surface 14 of the adjustable absorbent article 10. Alternatively, glue-like substances may be extracted from the herbaceous plant to be used as an adhesive for the adjustable absorbent article 10.

If included, the hemp-based coating and/or oil may be included in an amount of up to 1, 2, 3, 4, 5, 6, 7, 8, 9, 10, 20, or 30, % by weight based on the total weight of the absorbent article 10. Likewise, hemp-based coating and/or oil may be included in an amount of from 0.1 to 30, 0.5 to 10, 0.5 to 5, % by weight based on the total weight of the absorbent article 10. The amount of the hemp-based coating and/or oil may vary outside of the ranges described above. In additional non-limited embodiments, all values and ranges of values within are hereby expressly contemplated.

In one configuration, the absorbent article 10 may be coated or infused with a cannabinoid extracted from a Cannabis plant. Cannabinoids are naturally occurring chemical compounds derived from the Cannabis plant and include, but are not limited to, cannabidiol (CBD), cannabinol (CBN), cannabigerol (CBG), and cannabichromene (CBC). These compounds have many medicinal and therapeutic effects of Cannabis, which each compound offering distinctive properties and benefits. CBD, CBN, CBG, and CBC have little or no psychoactive effects and can be used to medicinal and therapeutic purposes without getting a high. Some cannabinoid compounds may be transformed into oils, pills, creams and more to suit various medicinal and/or therapeutic needs. It is contemplated that other types or class of cannabinoids may be infused, applied, provided, and the like.

There are many advantages of using cannabinoids in absorbent articles as they have non-psychoactive, medicinal, and/or therapeutic effects. Cannabinoids can help regulate sleep, immune-system responses, inflammation, anxiety, eczema, acne, and pain and maintain a pH balance. Other advantages and benefits of cannabinoids are contemplated. Further, CBN may be provided for its anti-inflammatory properties and can act as a pain reliever when combined with CBD. The presence of CBD helps balance effects of tetrahydrocannabinol (THC), such as paranoia and anxiety, and the presence of CBN produces a sedative that may be beneficial for sleeping. Further, the presence of CBG helps regulate sleep, mood, and appetite and the presence of CBC helps fight diseases. Additionally, CBC may be useful with pain relief, anti-viral, anti-bacterial, and anti-fungal.

If included, the cannabinoid coating or infusion may be included in an amount of up to 1, 2, 3, 4, 5, or 10, % by weight based on the total weight of the absorbent article 10. Likewise, cannabinoid coating or infusion may be included in an amount of from 0.01 to 10, 0.01 to 5, 0.01 to 2, 0.01 to 1, % by weight based on the total weight of the absorbent article 10. The amount of cannabinoid may vary outside of the ranges described above. In additional non-limited embodiments, all values and ranges of values within are hereby expressly contemplated.

Alternatively, the amount of cannabinoid present in the absorbent article 10 may vary over a wide range, but by way of example, often ranges from about 1 to about 300 mg, more usually from about 2 to about 250 mg, and typically from about 3 to about 200 mg, about 4 to about 180 mg, or about 5 to about 160 mg. It is contemplated that any amount of any type or class of cannabinoid may be present. The amount of cannabinoid may vary outside of the ranges described above. In additional non-limited embodiments, all values and ranges of values within are hereby expressly contemplated.

In some configurations, CBD oil may be extracted and applied to the absorbent article 10 and may be a naturally occurring and/or synthetically prepared compound. For example, the CBD oil may be applied under conditions sufficient to infuse the CBD oil into the plurality of layers 22, for therapeutic remedies including, but not limited to, healing and relieving the wearer of any menstrual pain or cramping. In another example, the CBD oil may be applied to a surface of each layer of the plurality of layers 22. In yet another example, the CBD oil may be applied under conditions sufficient to infuse the CBD oil between the plurality of layers 22. It will be appreciated that the CBD oil may be infused and/or applied to the absorbent article 10 in any suitable manner. It is contemplated that any type or class of cannabinoids may infused, applied, combined with, and the like to the absorbent article 10.

The absorbent article 10 may include other aesthetic and therapeutic properties to result in less fouling, smell, pain, abrasion or harm of the wearer. For example, an essential oil may be infused in the absorbent article 10 for therapeutic remedies. Other types of oil may be infused in or applied to any portions of the absorbent article 10. In yet another example, the absorbent article 10 may include any type of lotion composition. In a preferred configuration, the absorbent article 10 may include a hemp-based lotion composition. The lotion may be applied in any type of manner, pattern, and/or amount. The lotion may be applied with small droplets, discrete dots, stripes that run along the extension of the plurality of layers 22 and/or any portions of the absorbent article 10. It will be appreciated that any oil, lotion or the like may be applied to the absorbent article 10 in different amounts, locations, patterns of distribution, and/or methods. It will further be appreciated that any oil, lotion, or the like may be applied to the absorbent article 10 for therapeutic remedies including, but not limited to, healing, moisturizing, or relieving the wearer.

Referring back to FIGS. 1A-2, the adjustable absorbent article 10 includes at least one perforation line 24. The at least one perforation line 24 delineates at least two portions of the absorbent article 10. For example, as shown in FIG. 1A, the perforation lines 24A, 24B may round the left and right side of the center portion 18. In this configuration, the perforation lines 24A, 24B delineate the first side portion 20A and the second side portion 20B from the center portion 18. In another example, a perforation line 24 may be disposed along the entire perimeter of the center portion 18. In this configuration, the perforation line 24 may delineate a single side portion 20 from the center portion 18. In yet another example, two or more perforation lines 24 may be disposed along the entire perimeter of the center portion 18. In this configuration, the perforation line 24 may delineate two or more side portions 20. It will be appreciated that perforations may vary in size, shape, length, and width.

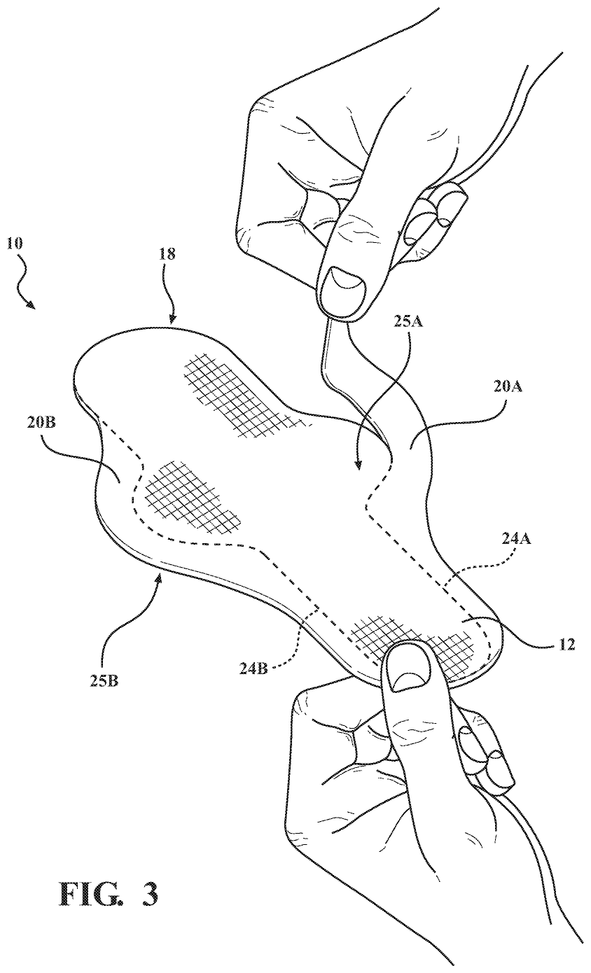

The perforation line 24 extends through the adjustable absorbent article 10 such that the adjustable absorbent article 10 allows the wearer to adjust the shape (including size and/or dimension) of the adjustable absorbent article 10 at the wearer's discretion. If the wearer decides to change the type of undergarment currently being worn or change the coverage of the adjustable absorbent article 10, the wearer may adjust the absorbent article 10 to match the type of undergarment or fit of the adjustable absorbent article 10. FIGS. 3-5 illustrate an exemplary adjustment of the absorbent article 10.

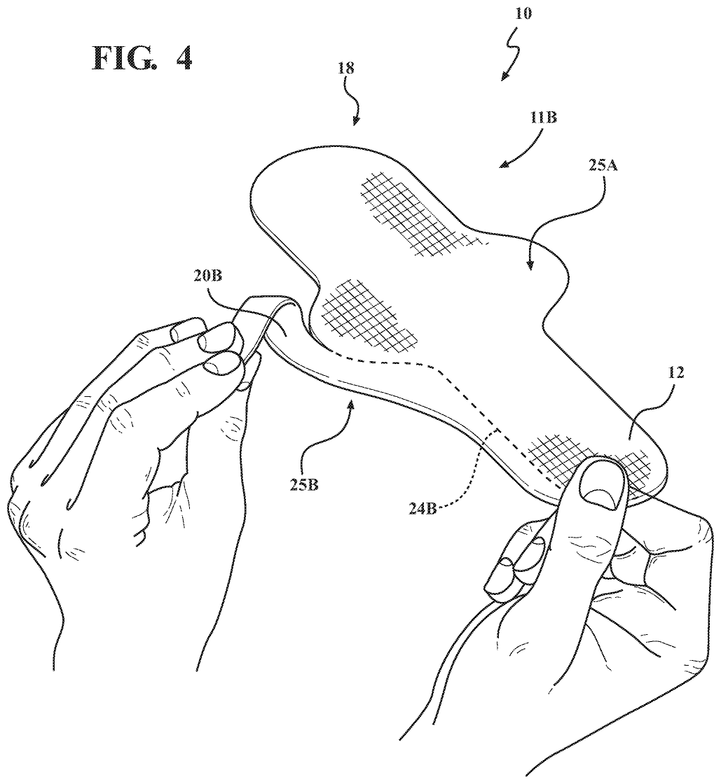

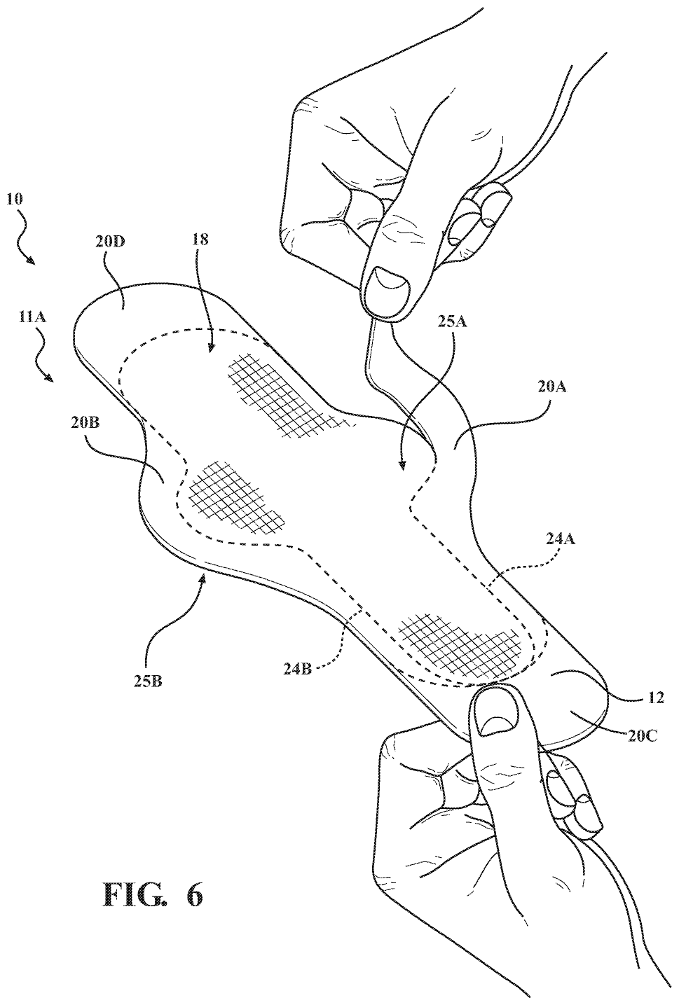

Referring to FIGS. 3-4 and 6-9, the adjustable absorbent article 10 has an adjustable profile 11. If the wearer decides to change the type of clothing currently being worn or change the coverage of the adjustable absorbent article 10, the wearer may adjust the absorbent article 10 to match the type of undergarment or fit of the adjustable absorbent article 10. In some configurations, the absorbent article 10 includes an initial profile 11A and a second profile 11B, as shown in FIGS. 3-4. In these configurations, the initial profile includes a first width and the second profile includes a second width. Further, in these configurations, the initial profile 11A is a full-size configuration including a first width. The second profile 11B is a thong configuration including a second width. The first width of the full-size configuration is greater than the second width of the thong configuration. In other words, the second width is configured to be narrower than the first width. The full-size configuration is provided for larger or full-sized clothing (e.g., undergarments, panties, swimsuits, one-pieces) and the thong configuration is provided for smaller sized clothing (e.g., thongs, swimsuits). In other configurations, the absorbent article 10 includes two or more profiles, as shown in FIGS. 6-8. For example, the absorbent article 10 includes an initial profile 11A, a second profile 11B, a third profile 11C, and a fourth profile 11D. It is contemplated that the absorbent article 10 includes any number of profiles.

FIGS. 3 and 4 are top views illustrating adjustment of the absorbent article 10 with side portions 20A, 20B being torn away from the center portion 18 along the perforated lines 24A, 24B are illustrated. If the wearer decides to change from a full-size undergarment to a smaller size undergarment (e.g. a thong or even a swim suit), the wearer may tear the side portions 20A, 20B along the perforated lines 24A, 24B to separate the side portions 20A, 20B, thereby adjusting the size of the adjustable absorbent article 10. In this configuration, the wearer may grasp the center portion 18 with one hand and with the other hand, tear each side portion 20A, 20B away.

FIGS. 5A and 5B are a top and bottom view, respectively, of an exemplary full-size configuration of the adjustable absorbent article, according to another configuration. In this configuration, the absorbent article 10 includes two or more detachable side portions 20. More specifically, the side portions are shown as 20A, 20B, 20C, and 20D. The two or more side portions 20 are detachable. Shown in FIG. 5B, a sixth adhesive portion 16F may be disposed on the bottom surface 14 of the side portion 20D and a seventh adhesive portion 16G may be disposed on the bottom surface 14 of the side portion 20C. It is contemplated that the sixth and seventh adhesive portions 16F, 16G may be sized and shaped according to usage. For example, the sixth and seventh adhesive portions 16F, 16G may line the perimeter of the side portions 20D, 20C, respectively.

Similar to FIGS. 3 and 4, FIGS. 6-9 are top views illustrating adjustment of the absorbent article 10 with side portions 20A, 20B, 20C, and 20D being torn away from the center portion 18. The adjustable absorbent article 10 includes an initial profile 11A, a second profile 11B, a third profile 11C, and a fourth profile 11D. The initial profile 11A includes all the side portions 20A, 20B, 20C, and 20D in a full-size configuration. As mentioned above, the full-size configuration includes a first width. To convert between adjustable profiles 11, the wearer may tear one or more of the side portions 20A, 20B, 20C, or 20D. The one or more side portions 20A, 20B, 20C, and 20D are detachable along a perforated line.

Referring to FIGS. 6-9, one exemplary adjustment is provided. The wearer may adjust the width of the absorbent article 10. In FIGS. 6 and 7, the wearer tears two side portions (20A and 20B) along the perforation lines 24A and 24B, respectively, to convert the adjustable absorbent article 10 from initial profile 11A to the second profile 11B. In this configuration, the initial profile includes a first width and the second profile includes a second width. Further, in this configuration, the initial profile 11A is a full-size configuration including the first width. The first width is from one end of the winged portion 20B to another end of the winged portion 20B of the side portion 20A. To put it differently, the first width is from an outer edge of the second side portion 20B to an outer edge of the first side portion 20A. The second profile 11B is a thong configuration including the second width. The second width is from an edge of the winged portion 25A of the center portion 18 to another edge of the winged portion 25A. The first width of the full-size configuration is greater than the second width of the thong configuration.

The wearer may adjust the length of the absorbent article 10. FIG. 8 is illustrating the wearer tearing one side portion 20C to convert the adjustable absorbent article 10 from the second profile 11B to a third profile 11C. The second profile 11B may include a first length and the third profile 11C may include a second length. The first length is from the third side portion 20C to the fourth side portion 20D. The second length is from the third side portion 20C to one end of the center portion 18. The first length of the second profile 11B is greater than the second length of the third profile 11C.

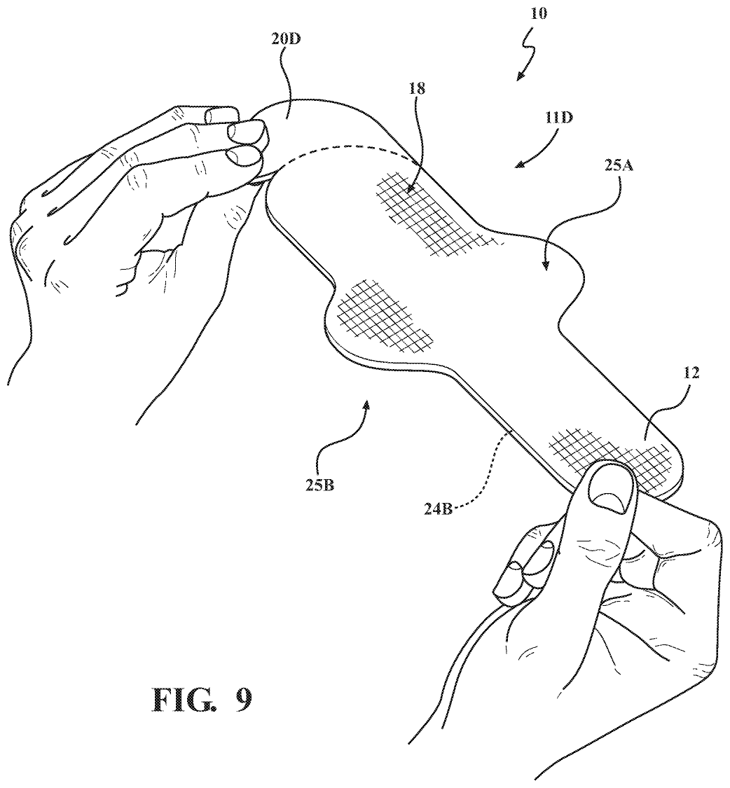

FIG. 9 is illustrating the wearer tearing one side portion 20D to convert the adjustable absorbent article 10 from the third profile 11C to the fourth profile 11D. The fourth profile 11D may include a third length. The third length is from one end of the center portion 18 to another end of the center portion 18. The second length of the third profile 11C is greater than the third length of the fourth profile 11D.

In another exemplary adjustment, the wearer may tear side portion 20C to convert the initial profile 11A (including all side portions 20) to the second profile 11B. In yet another exemplary adjustment, the wearer may tear side portions 20C and 20D to convert the initial profile 11A (including all side portions 20) to the second profile 11B. It is contemplated that the wearer may tear any number of side portions to convert the absorbent article 10 to different profiles. Other adjustments to the length and width of the absorbent article is contemplated.

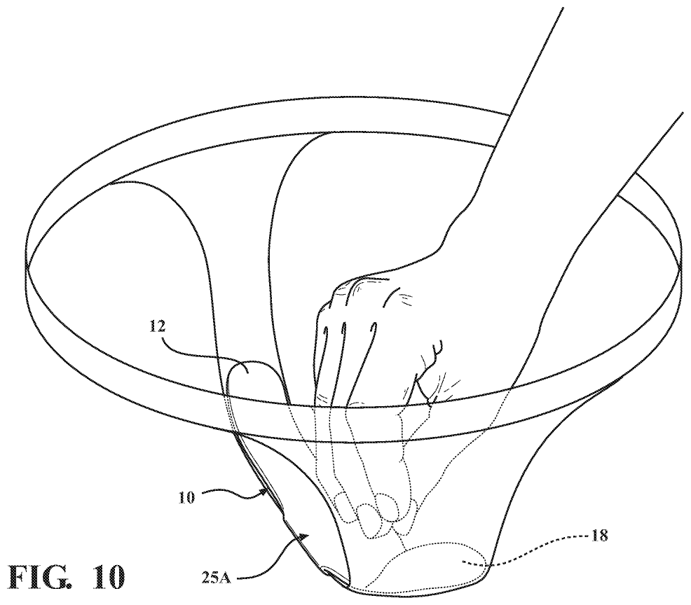

Referring to FIG. 10, a top view of an exemplary thong configuration of the adjustable absorbent article 10 being attached to a wearer's undergarment is illustrated. In the thong configuration, once the side portions 20A, 20B are torn away, the wearer may attach the adjustable absorbent article 10 to a thong. The adhesive portions 16 may aid in the attachment of the adjustable absorbent article 10. Other methods of attachment are contemplated.

It is also contemplated that the wearer may adjust any shape, size, thickness, and/or dimension of the adjustable absorbent article 10. For example, the wearer may tear along a perforated line to separate a side portion 20 to adjust the length of the adjustable absorbent article 10. In this configuration, the wearer may make the adjustable absorbent article 10 shorter by tearing a side portion 20 attached to a proximal end or a distal end of the adjustable absorbent article 10. In another example, the wearer may tear along a perforation line 24 to separate a side portion 20 to adjust the thickness of the adjustable absorbent article 10. In this configuration, one or more of the plurality of layers of the center portion 18 may extend to the side portion 20.

The wearer may adjust shape of the adjustable absorbent article 10 using various methods. For example, the wearer may use a tool, such as scissors, to separate the side portions 20A, 20B away from the center portion 18. Other methods of adjustment include, but are not limited to, peeling, pulling, folding or the like.

It will be further appreciated that the terms “include,” “includes,” and “including” have the same meaning as the terms “comprise,” “comprises,” and “comprising.”

Several embodiments have been discussed in the foregoing description. However, the embodiments discussed herein are not intended to be exhaustive or limit the disclosure to any particular form. The terminology which has been used is intended to be in the nature of words of description rather than of limitation. Many modifications and variations are possible in light of the above teachings and the disclosure may be practiced otherwise than as specifically described.

What is claimed is:

1. An adjustable absorbent article comprising:
a center portion including a plurality of layers for absorption of fluid;
a side portion delineated from the center portion by at least one perforation line and positioned adjacent the center portion, the at least one perforation line extends through the adjustable absorbent article allowing the absorbent article to be adjustable by a wearer,
wherein the side portion is removable from the center portion by tearing along the at least one perforation line for allowing a wearer to adjust the absorbent article; and
an adhesive portion disposed on the center portion for adhering to an undergarment of the wearer,
wherein the center portion, side portion, adhesive portion or any combinations thereof comprises hemp.

2. The adjustable absorbent article of claim 1, wherein hemp includes hemp fiber, hemp oil, hemp paste, hemp extract, hemp probiotic, hemp remedy, hemp essence and combinations thereof.

3. The adjustable absorbent article of claim 1, wherein one or more layers of the center portion is formed of hemp fiber and/or a hemp-based polymer.

4. The adjustable absorbent article of claim 1, wherein the side portion is formed of hemp fiber and/or a hemp-based polymer.

5. The adjustable absorbent article of claim 1, wherein the adhesive portion comprises hemp oil, hemp paste, hemp essence, and/or combinations thereof.

6. The adjustable absorbent article of claim 1, wherein the center portion and/or the side portion comprises greater than 50% by weight hemp fiber, based on a total weight of the center portion and/or the side portion.

7. The adjustable absorbent article of claim 1, wherein the center portion includes a top layer, a core layer, and a bottom layer, wherein the bottom layer is impervious.

8. The adjustable absorbent article of claim 7, wherein the bottom layer comprises a biopolymer.

9. The adjustable absorbent article of claim 7, wherein the bottom layer comprises a water-dispersible and/or a biodegradable polymer.

10. The adjustable absorbent article of claim 7, wherein the top layer, the core layer, and the bottom layer are fixedly secured to the preceding one of the plurality of layers including the top, core, and/or bottom layer.

11. The adjustable absorbent article of claim 1, having an initial profile and a second profile, wherein the side portion is tearable from the center portion to convert the initial profile to the second profile.

12. The adjustable absorbent article of claim 11, wherein the initial profile is a full-size configuration including a first width and the second profile is a thong configuration including a second width.

13. The adjustable absorbent article of claim 12, wherein the side portion includes a winged portion and in the full-size configuration, the winged portion is configured to wrap around the undergarment to provide stability.

14. The adjustable absorbent article of claim 12, wherein the center portion includes a winged portion and in the thong configuration, the winged portion is configured to wrap around the undergarment to provide stability.

15. The adjustable absorbent article of claim 1, having an initial profile and a second profile, wherein the second profile includes a first width and a second width, the second width configured to be narrower than the first width.

16. The adjustable absorbent article of claim 1, further comprising two or more side portions positioned adjacent the center portion, with a first and a second side portion delineated from the center portion by at least one perforation line and positioned adjacent the center portion, the at least one perforation line extends through the adjustable absorbent article allowing the absorbent article to be adjustable by a wearer.

17. The adjustable absorbent article of claim 16, further comprising two or more profiles, wherein the two or more side portions are tearable from the center portion to convert an initial profile to a second profile.

18. The adjustable absorbent article of claim 17, wherein the two or more side portions are tearable from the center portion to convert the second profile to a third profile.

19. The adjustable absorbent article of claim 17, wherein one of the two or more profiles is a full-size configuration and one of the two or more profiles is a thong configuration.

20. The adjustable absorbent article of claim 19, wherein a first side portion and a second side portion each include a first and second winged portion, respectively, and in the full-size configuration, the first and second winged portions are configured to wrap around the undergarment to provide stability.

21. The adjustable absorbent article of claim 17, wherein the two or more side portions comprises a plurality of layers, including at least one impervious layer.

22. The adjustable absorbent article of claim 1, further including a coating or infusion comprising a cannabinoid.
